(19) United States
(12) Patent Application Publication
(10) Pub. No.: US 2006/0003344 A1
(43) Pub. Date: Jan. 5, 2006

(54) Methods related to a single nucleotide polymorphism of the G protein coupled receptor, GPR40

(75) Inventors: Karen L. Houseknecht, Old Saybrook, CT (US); Poulabi Banerjee, Mystic, CT (US)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 11/046,020

(22) Filed: Jan. 28, 2005

Related U.S. Application Data
(60) Provisional application No. 60/584,686, filed on Jun. 30, 2004.

(51) Int. Cl. C12Q 1/68

(52) U.S. Cl. 435/6

(57) ABSTRACT

This invention relates to genotyping methods, methods of treatment, diagnostic tests and kits and methods of characterizing an agent, related to a single nucleotide polymorphism of the GPR40 gene.

### FIELD OF THE INVENTION

This invention relates to genotyping methods, methods of treatment, diagnostic tests and kits and methods of characterizing an agent, related to a single nucleotide polymorphism of the GPR40 gene.

### BACKGROUND OF THE INVENTION

Diabetes is a serious and sometimes fatal disease that affects over 17 million Americans and 151 million people worldwide. Gadsby (2002). Diabetes results from the body's failure to produce or properly respond to insulin. Glucose is the major substrate that regulates insulin secretion but other nutrients like free fatty acids (FFA) and amino acids can modify insulin secretion. Haber et al. (2002).

There are two major types of diabetes. In Type I diabetes, insulin producing pancreatic β-cells are destroyed by the body's own immune system, leading to insulin deficiency. Type II diabetes results from the loss of sensitivity by the cells of the body to the action of insulin and the inability of the pancreas to release insulin appropriately. The incidence of Type II diabetes is increasing, and, in the U.S., has become especially prevalent in certain at risk populations. Gadsby (2002).

Metabolic syndrome is a clustering of metabolic conditions that increases the risk for developing diabetes and cardiovascular disease. Park et al. (2003). Conditions including obesity, insulin resistance, dyslipidemia, and hypertension have been reported as being key components of metabolic syndrome. Reilly and Rader (2003); Park et al. (2003); Expert Panel on Detection, Evaluation, and Treatment of High Blood Cholesterol in Adults (2001).

Obesity has been identified as a core factor contributing to metabolic syndrome. It has been proposed that a reduction in excess body weight would minimize the risks associated with metabolic syndrome. Shirai (2004).

The G-protein coupled receptors (GPCRs) are a superfamily of membrane proteins characterized by the presence of seven transmembrane α-helix segments (designated TM1 to TM7) connecting alternating intracellular and extracellular loops. Gether (2000). GPCRs mediate signaling between signal molecules, such as hormones, neurotransmitters and local mediators, and intracellular enzymes, ion channels and transporters. See Johnson and Dhanasekaran (1989).

Sawzdargo et al. (1997) disclose the identification of the GPCR gene, GPR40, localized on chromosome 19q13.1, encoding a 300 amino acid protein and identified by GenBank® accession number AF024687.

Haga et al. (2002) disclose 190,562 genetic variations of the human genome, including a single nucleotide polymorphism of the GPR40 gene in which nucleotide 632 of the coding sequence (corresponding to nucleotide 642 in the sequence of GenBank accession number AF024687) may be either guanine (G) or adenosine (A), such that amino acid 211 is either arginine or histidine. This single nucleotide polymorphism is identified in the National Center for Biotechnology Information (NCBI) dbSNP database as accession number rs2301151.

International Patent Application Publication No. WO 02/057783 and Briscoe et al. (2003) disclose that GPR40 is specifically expressed in pancreatic β-cells as well as in brain of the human, mouse and rat. Furthermore, WO 02/057783 and Briscoe et al. identify medium and long chain saturated and unsaturated fatty acid ligands of GPR40 from experiments measuring calcium ion concentration.

Kotarsky et al. (2003) disclose that GPR40 is activated, not only by a range of fatty acids, but also by antidiabeteic thiazolidinedione drugs, as measured by a reporter system linked to the GPR40 receptor.

Itoh et al. (2003) disclose that binding and activation of GPR40 by free fatty acids increases insulin secretion.

International Patent Application Publication No. WO 04/072650 disclose nucleic acid sequences and amino acid sequences of human GPR40 and its regulation for the treatment of hematological diseases, disorders of the peripheral and central nervous system, gastrointestinal diseases, respiratory diseases, metabolic diseases, cancer, cardiovascular diseases, and urological diseases in mammals.

### SUMMARY OF THE INVENTION

The present invention relates, in part, to genotyping methods, comprising determining the amino acid that is encoded by the GPR40 gene of a human subject at amino acid number 211. In a preferred embodiment, the amino acid encoded at amino acid number 211 of both alleles of the GPR40 gene is determined. In another preferred embodiment, said genotyping methods involve determining whether said amino acid number 211 is other than histidine. In a further preferred embodiment, said genotyping methods involve determining whether said amino acid number 211 is arginine. In an additional preferred embodiment, said genotyping methods involve determining whether said amino acid number 211 is histidine. In a still further preferred embodiment, said genotyping methods comprise, isolating nucleic acid from a human subject, amplifying a contiguous sequence of said nucleic acid, wherein said contiguous sequence comprises the nucleotides of GPR40 that encode amino acid number 211, or the complementary sequence thereof, treating said contiguous sequence with a nucleotide probe that enables determination of the amino acid encoded at amino acid number 211 of the GPR40 gene of said subject; and determining whether said probe hybridizes to said contiguous sequence under conditions of high stringency. In a preferred embodiment, said probe consists of a contiguous portion of a nucleotide sequence of SEQ. ID. NO: 9 or of SEQ. ID. NO: 10 having a length of about 13 to about 35 nucleotides, linked to a detectable label, and including the nucleotides encoding amino acid 211, or the complementary nucleotide sequence thereof.

A further aspect of the invention provides methods of treatment, comprising, determining the amino acid that is encoded by the GPR40 gene of a human subject at amino acid number 211 and treating said subject with a therapeutically effective amount of an Agent for treatment or prevention of a disease or pathological condition mediated by having at least one allele, and preferably both alleles, of the GPR40 gene wherein the encoded amino acid number 211 is other than histidine, preferably arginine. In a preferred embodiment, said Agent is selected from insulin, an insulin secretion stimulating sulfonylurea compound, a glycogen phosphorylase inhibitor, a biguanide hepatic glucose output inhibitor, an alpha-glucosidase inhibitor, a protein tyrosine phosphatase-1B inhibitor, a dipeptidyl peptidase IV inhibitor, a glycogen synthase kinase-3 beta inhibitor, a peroxisome proliferator-activated receptor gamma agonist, a glucagon receptor antagonist, a selective serotonin reuptake inhibitor, a 3-hydroxy-3-methylglutaryl coenzyme A reductase inhibitor, a gamma-aminobutyric acid agonist, an angiotensin converting enzyme inhibitor, an angiotensin-II receptor antagonist, a phosphodiesterase type 5 inhibitor, a sorbitol dehydrogenase inhibitor and an aldose reductase inhibitor, a pharmaceutically acceptable prodrug thereof or pharmaceutically acceptable salt thereof. In another preferred embodiment, said Agent is an anti-obesity agent, preferably selected from an apolipoprotein-B secretion/microsomal triglyceride transfer protein inhibitor, an 11β-hydroxy steroid dehydrogenase-1 inhibitor, a peptide YY₃₋₃₆, an analog of a peptide YY₃₋₃₆, a cannabinoid antagonist, an MCR-4 agonist, a cholecystokinin-A agonist, a monoamine reuptake inhibitor, a sympathomimetic agents, a P3 adrenergic receptor agonist, a dopamine agonist, a melanocyte-stimulating hormone receptor analog, a 5HT2c agonist, a melanin concentrating hormone antagonist, leptin, a leptin analog, a leptin receptor agonist, a galanin antagonist, a lipase inhibitor, an anorectic agent, a neuropeptide-γ receptor antagonist, a thyromimetic agent, dehydroepiandrosterone, an analog of dehydroepiandrosterone, a glucocorticoid receptor agonist, a glucocorticoid receptor antagonist, an orexin receptor antagonist, a glucagon-like peptide-1 receptor agonist, a ciliary neurotrophic factor, a human agouti-related protein, a ghrelin receptor antagonist, a histamine 3 receptor antagonist, a histamine 3 receptor inverse agonist and a neuromedin U receptor agonist.

Another aspect of the invention relates to kits, comprising a nucleotide probe that enables determination of the amino acid encoded at amino acid number 211 of the GPR40 gene of a human subject. In a preferred embodiment, said probe enables determination whether amino acid number 211 of said GPR40 gene is histidine. In another embodiment, said probe enables determination whether amino acid number 211 of said GPR40 gene is other than histidine, preferably arginine. In a further preferred embodiment, said probe consists of a contiguous portion of a nucleotide sequence of SEQ. ID. NO: 9 or of SEQ. ID. NO: 10 having a length of about 13 to about 35 nucleotides, linked to a detectable label, and including the nucleotides encoding amino acid 211, or the complementary nucleotide sequence thereof. In an additional preferred embodiment, said kits further comprise either: a first polymerase chain reaction nucleotide primer that is complementary to a nucleotide sequence that is downstream from the nucleotides encoding amino acid 211 of said GPR40 gene and a second polymerase chain reaction primer that is complementary to a nucleotide sequence that is upstream from the nucleotides encoding said amino acid 211; or, a first primer that is complementary to a nucleotide sequence that is upstream from the nucleotides encoding said amino acid 211 and a second polymerase chain reaction primer that is complementary to a nucleotide sequence that is downstream from the nucleotides encoding said amino acid 211. In another preferred embodiment said kits further comprise a container.

A further aspect of the invention relates to methods of characterizing an Agent, comprising measuring the competitive inhibition by a test Agent of binding of a GPR40 ligand to a first human GPR40 receptor wherein amino acid number 211 is histidine and measuring the competitive inhibition by said compound of binding of a GPR40 ligand to a second human GPR40 receptor wherein amino acid number 211 is other than histidine. In a preferred embodiment, amino acid number 211 of said second receptor is arginine. In a further preferred embodiment, the methods further comprise comparing the competitive inhibition of binding by said compound of said first receptor and said second receptor.

An additional aspect of the invention relates to methods of characterizing an Agent, comprising, measuring the activation by a test Agent of a first human GPR40 receptor wherein amino acid number 211 is histidine and measuring the activation by a test Agent of a second human GPR40 receptor wherein amino acid number 211 is other than histidine. In a preferred embodiment, the amino acid number 211 of said second receptor is arginine. In a further preferred embodiment, said methods further comprise comparing the activation by said compound of said first receptor and said second receptor.

Another aspect of the invention provides methods of characterizing an Agent, comprising measuring the inhibition by a test Agent of activation of a first human GPR40 receptor by an activator of GPR40, wherein amino acid number 211 of said first receptor is histidine and measuring the inhibition by a test Agent of activation of a second human GPR40 receptor by an activator of GPR40, wherein amino acid number 211 of said second receptor is other than histidine, preferably, arginine. In a further preferred embodiment, said methods further comprise comparing the inhibition of activation by said compound of said first receptor and said second receptor.

### BRIEF DESCRIPTION OF THE DRAWINGS

FIG. 1 illustrates the activation of GPR40 by thiazolidinedione compounds as measured by intracellular calcium ion release in GPR40 expressing HEK 293 cells as compared to HEK 293 cells that do not express GPR40.

FIG. 2 shows a comparison between the activation by thiazolidinedione compounds of GPR40 having histidine versus arginine at amino acid 211 as measured by intracellular calcium ion release in GPR40 expressing HEK 293 cells.

FIG. 3 shows logistic regression curves (solid lines) with 95% confidence limits (dotted lines) and individual data points for each genotype of GPR40 in a population of 2465 subjects (1332 male and 857 female) comparing body mass index to the likelihood of having diabetes. The data points at the top of the plot indicate the body mass index of individuals with diabetes. The data points at the bottom of the plot indicate the body mass index of individuals who do not have diabetes.

FIG. 4 shows a restriction map of the expression vector used to prepare GPR40 expressing HEK 293 cells.

FIG. 5 shows nucleotide and amino acid sequences (SEQ. ID. NO:1 and 2 respectively) that encodes human GPR40 wherein the nucleotides that encode amino acid number 211 and amino acid number 211 as arginine are highlighted.

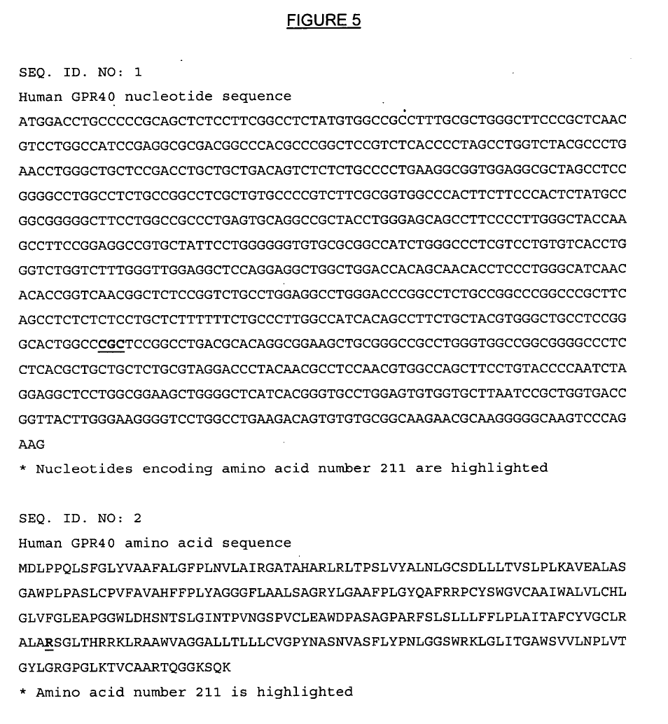

FIG. 6 shows nucleotide and amino acid sequences (SEQ. ID. NO:3 and 4 respectively) that encodes human GPR40 wherein the nucleotides that encode amino acid number 211 and amino acid number 211 as histidine are highlighted.

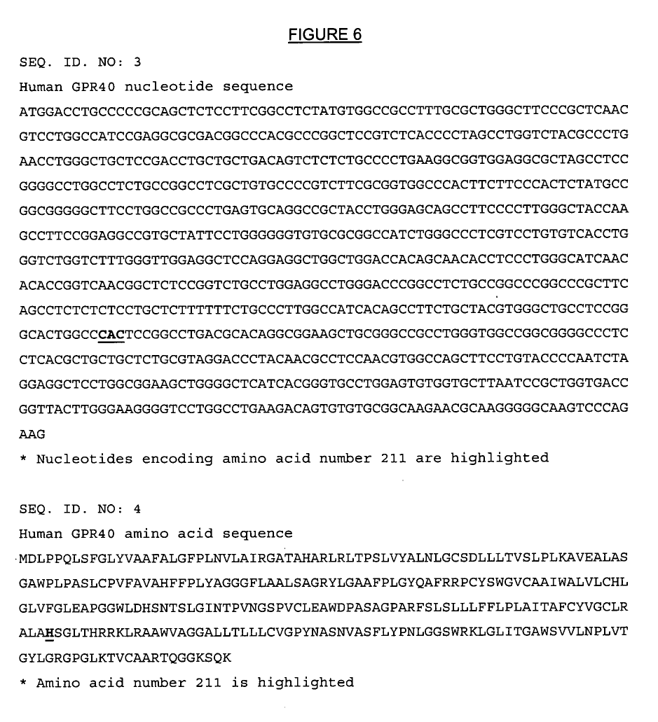

FIG. 7 shows the sequences of PCR primers and PCR probes that may be used to amplify and detect sequences containing the GPR40 alleles. FIG. 7 also shows nucleotide sequences encoding amino acid number 211 as either histidine (SEQ. ID. NO: 9) or arginine (SEQ. ID. NO: 10).

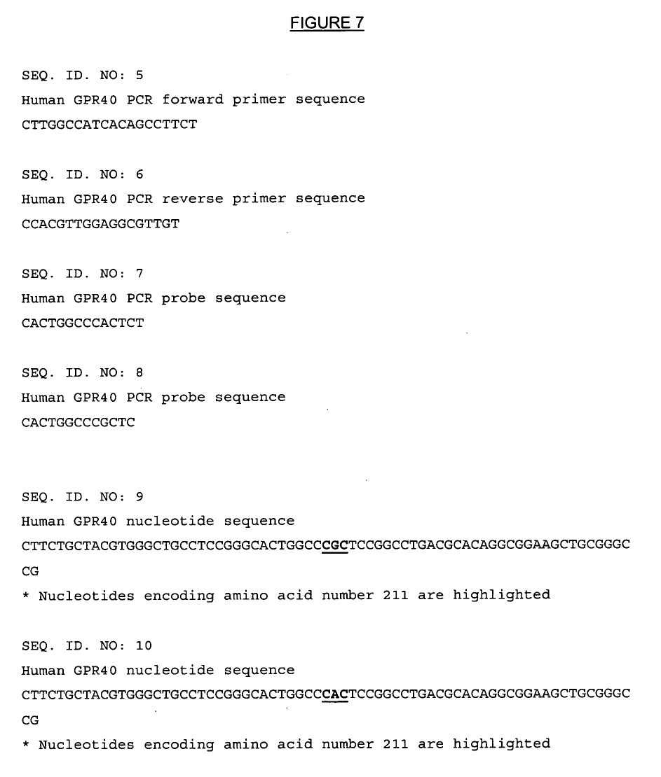

FIG. 8 shows the sequence of an expression vector construct containing a GPR40 allele that encodes histidine at amino acid number 211.

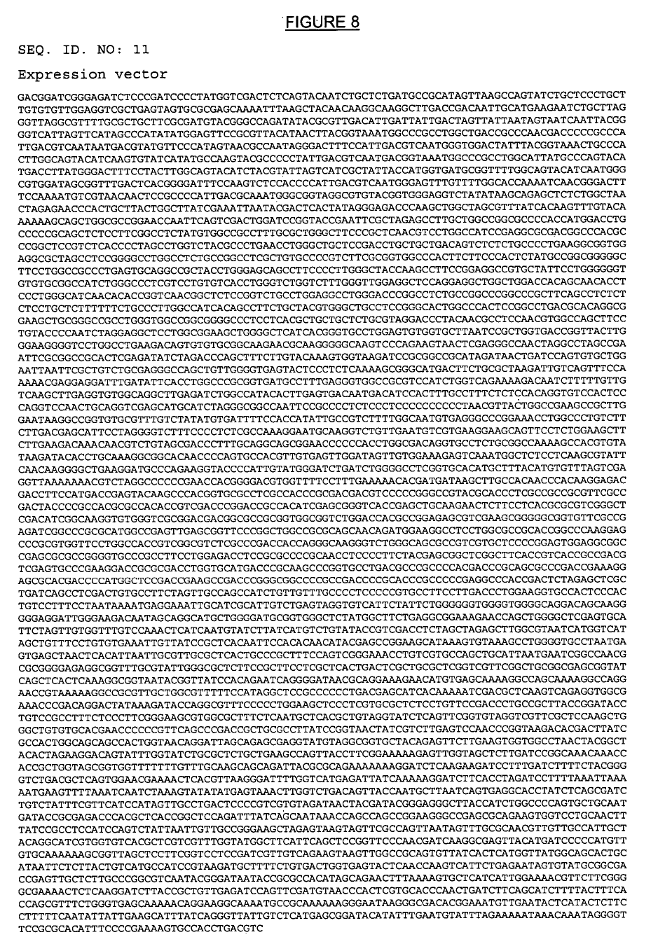

### DETAILED DESCRIPTION OF THE INVENTION

The terms used herein have their usual meaning in the art. However, to even further clarify the present invention and for convenience, the meaning of certain terms and phrases employed in the specification, including the examples and appendant claims are provided below.

The terms “Agent(s)” means substances, including peptides, and compounds for treatment or prevention of a disease or pathological condition that is mediated by having at least one allele, preferably both alleles, of the GPR40 gene wherein the encoded amino acid number 211 is other than histidine, preferably arginine, as further described herein.

The term “amino acid number 211” with respect to the GPR40 gene refers to amino acid residue number 211 in the sequence of GenBank accession number AF024687, also shown in SEQ. ID. Nos. 2 and 4.

The term “conditions of high stringency” means wash conditions of 68° C. in the presence of about 0.2×SSC and about 0.1% SDS, for 1 hour. Useful variations on these wash conditions will be readily apparent to those of ordinary skill in the art. Generally, stringency of hybridization is expressed, in part, with reference to the temperature under which the wash step is carried out. Generally, such wash temperatures are selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. An equation for calculating Tₘ and conditions for nucleic acid hybridization are well known and can be found in Sambrook (1989), at see volume 2, chapter 9.

The term “diabetes” means diabetes mellitus as understood by those of skill in the art. The term includes Type I diabetes mellitus, also known as insulin dependent diabetes mellitus (IDDM), and Type II diabetes mellitus, also known as non-insulin dependent diabetes mellitus (NIDDM). The World Health Organization (W.H.O.) has suggested criteria for diagnosing diabetes mellitus (W.H.O. 1980/85 Technical Report Series No. 646/727). Diabetes has also been characterized by the National Diabetes Data Group (see National Diabetes Data Group: Classification and diagnosis of diabetes mellitus and other categories of glucose intolerance, Diabetes, 28:1039-1044, 1979). However, more recently, guidelines have been set forth, for example by the American Diabetes Association, to enable diagnosis of Type I and Type II diabetes (see, for example, ADA (2004)).

The term “therapeutically effective amount” means an amount of an agent that (i) treats or prevents a particular disease, condition or disorder; (ii) attenuates, ameliorates or eliminates one or more symptoms of a particular disease, condition or disorder; or (iii) prevents or delays the onset of one or more symptoms of a particular disease, condition or disorder. A therapeutically effective amount can be determined by one of ordinary skill in the art, based upon the present disclosure, on an individual basis and will be based, at least in part, on considerations of the species of the subject, the size of the subject, the type of delivery system used, and the type of administration relative to the progression of the disease.

The expression “pharmaceutically acceptable salts” includes both pharmaceutically acceptable acid addition salts and pharmaceutically acceptable cationic salts, where appropriate. Pharmaceutically acceptable cationic salts include, but are not limited to, salts such as the alkali metal salts, (e.g., sodium and potassium), alkaline earth metal salts (e.g., calcium and magnesium), aluminum salts, ammonium salts, and salts with organic amines such as benzathine (N,N′-dibenzylethylenediamine), choline, diethanolamine, ethylenediamine, meglumine (N-methylglucamine), benethamine (N-benzylphenethylamine), diethylamine, piperazine, tromethamine (2-amino-2-hydroxymethyl-1,3-propanediol) and procaine. The expression “pharmaceutically-acceptable acid addition salts” is intended to include, but is not limited to, such salts as the hydrochloride, hydrobromide, sulfate, hydrogen sulfate, phosphate, hydrogen phosphate, dihydrogenphosphate, acetate, succinate, citrate, methanesulfonate (mesylate) and p-toluenesulfonate (tosylate) salts. A particularly preferred salt is sodium salt. Descriptions of compounds appearing herein which include the phrase “prodrugs thereof or pharmaceutically acceptable salts thereof” or a substantially similar phrase are meant to include both pharmaceutically acceptable salts of the applicable compounds as well as pharmaceutically acceptable salts of such prodrugs.

The term “prodrug” means a compound that is transformed in vivo to yield a compound of the present invention. The transformation may occur by various mechanisms, such as through hydrolysis in blood. A discussion of the use of prodrugs is provided by Higuchi and Stella (1987). For example, when a compound of the present invention contains a carboxylic acid functional group, a prodrug can comprise an ester formed by the replacement of the hydrogen atom of the acid group with a group such as (C1-C8)alkyl, (C2-C12)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N-(C1-C2)alkylamino(C2-C3)alkyl (such as b-dimethylaminoethyl), carbamoyl-(C1-C2)alkyl, N,N-di(C1-C2)alkylcarbamoyl-(C1-C2)alkyl and piperidino-, pyrrolidino- or morpholino(C2-C3)alkyl. Similarly, when a compound of the present invention comprises an alcohol functional group, a prodrug can be formed by the replacement of the hydrogen atom of the alcohol group with a group such as (C1-C6)alkanoyloxymethyl, 1-((C1-C6)alkanoyloxy)ethyl, 1-methyl-1-((C1-C6)alkanoyloxy)ethyl, (C1-C6)alkoxycarbonyloxymethyl, N-(C1-C6)alkoxycarbonylaminomethyl, succinoyl, (C1-C6)alkanoyl, a-amino(C1-C4)alkanoyl, arylacyl and a-aminoacyl, or a-aminoacyl-a-aminoacyl, where each a-aminoacyl group is independently selected from the naturally occurring L-amino acids, P(O)(OH)₂, —P(O)(O(C1-C6)alkyl)₂ or glycosyl (the radical resulting from the removal of a hydroxyl group of the hemiacetal form of a carbohydrate). When a compound of the present invention comprises an amine functional group, a prodrug can be formed by the replacement of a hydrogen atom in the amine group with a group such as RX-carbonyl, RXO-carbonyl, NRXRX′-carbonyl where RX and RX′ are each independently (C1-C10)alkyl, (C3-C7)cycloalkyl, benzyl, or RX-carbonyl is a natural a-aminoacyl or natural a-aminoacyl-natural a-aminoacyl, —C(OH)C(O)OYX wherein YX is H, (C1-C6)alkyl or benzyl), —C(OYX0) YX1 wherein YX0 is (C1-C4) alkyl and YX1 is (C1-C6)alkyl, carboxy(C1-C6)alkyl, amino(C1-C4)alkyl or mono-N- or di-N,N-(C1-C6)alkylaminoalkyl, —C(YX2) YX3 wherein YX2 is H or methyl and YX3 is mono-N- or di-N,N-(C1-C6)alkylamino, morpholino, piperidin-1-yl or pyrrolidin-1-yl.

“Nucleotide sequence” and “polynucleotide” means DNA or RNA, whether in single-stranded or double-stranded form. The term “complementary nucleotide sequence” means a nucleotide sequence that anneals (binds) to a another nucleotide sequence according to the pairing of a guanidine nucleotide (G) with a cytidine nucleotide (C) and adenosine nucleotide (A) with thymidine nucleotide (T), except in RNA where a T is replaced with a uridine nucleotide (U) so that U binds with A.

The abbreviations used herein have their usual meaning in the art. However, to even further clarify the present invention, for convenience, the meaning of certain abbreviations are provided as follows: “° C.” means degrees centigrade; “CO₂” means carbon dioxide; “DMEM” means Dulbecco's modified Eagle's medium; “DNA” means deoxyribonucleic acid; “FBS” means fetal bovine serum; “g” means gram; “HEPES” means 4-(2-hydroxyethyl)-1-piperazineethanesulfonic acid; “kg” means kilogram; “mg” means milligram; “mL” means milliliter; “mM” means millimolar; “μl” means microliter; “μM” means microimolar; “ng” means nanogram; “nm” means nanometer; “nM” means nanomollar; “PCR” means polymerase chain reaction; “Pen/Strep” means a mixture containing penicillin and streptomycin; “RNA” means ribonucleic acid; “RPM” means revolutions per minute; and “xg” means times gravity.

The following amino acid abbreviations are used in this disclosure:

| | | | |
|---|---|---|---|
| A | Alanine | T | Threonine |
| V | Valine | C | Cysteine |
| L | Leucine | Y | Tyrosine |
| I | Isoleucine | N | Asparagine |
| P | Proline | Q | Glutamine |
| F | Phenylalanin | D | Aspartic Acid |
| W | Tryptophan | E | Glutamic Acid |
| M | Methionine | K | Lysine |
| G | Glycine | R | Arginine |
| S | Serine | H | Histidine |

The GPCR gene, GPR40, identified by GenBank accession number AF024687, encodes a 300 amino acid protein. Sawzdargo et al. (1997). GPR40, which is highly expressed in pancreatic islet cells has been linked to the regulation of insulin secretion and, thus, has been proposed as a target for the treatment of diabetes. Briscoe et al. (2003); Itoh et al. (2003). Kotarsky et al. (2003) propose that GPR40 mediates responses to thiazolidinedione-type diabetes drugs.

As with other GPCRs, GPR40 is involved in transmitting extracellular signals into the cell. Activation of GPR40 elicits release, via the pathway involving the G_{q} protein, of intracellular calcium. Briscoe et al. (2003). The resulting calcium flux is useful for detecting GRP40 activation by methods known to those of skill in the art based upon the present disclosure (see, for example: Kotarsky et al. (2003); Briscoe et al. (2003)).

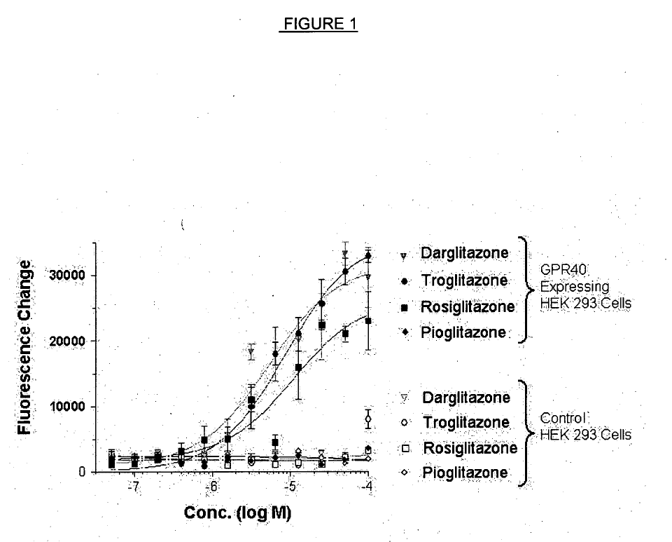

Thiazolidinedione and certain fatty acids are known to activate GPR40. Kotarsky et al. (2003) and Briscoe et al. (2003). As illustrated by FIG. 1, herein, thiazolidinedione compounds stimulate intracellular calcium ion release in GPR40 expressing cells as compared to cells that do not express GPR40.

This invention is based, in part, on the discovery that a single nucleotide polymorphism influences activation of GPR40, for example, by thiazolidinedione-type drugs. This polymorphism, which occurs at nucleotide 632 of the GPR40 coding sequence (corresponding to nucleotide 642 in the sequence of GenBank accession number AF024687), involves a substitution of A to a nucleotide other than A, preferably G, resulting in an amino acid substitution at residue 211 from histidine to an amino acid other than histidine, preferably arginine. Nucleotide and amino acid sequences of GPR40 wherein amino acid 211 is arginine appear at SEQ. ID. Nos. 1 and 2 respectively. Nucleotide and amino acid sequences of GPR40 wherein amino acid 211 is histidine appear at SEQ. ID. Nos. 3 and 4 respectively.

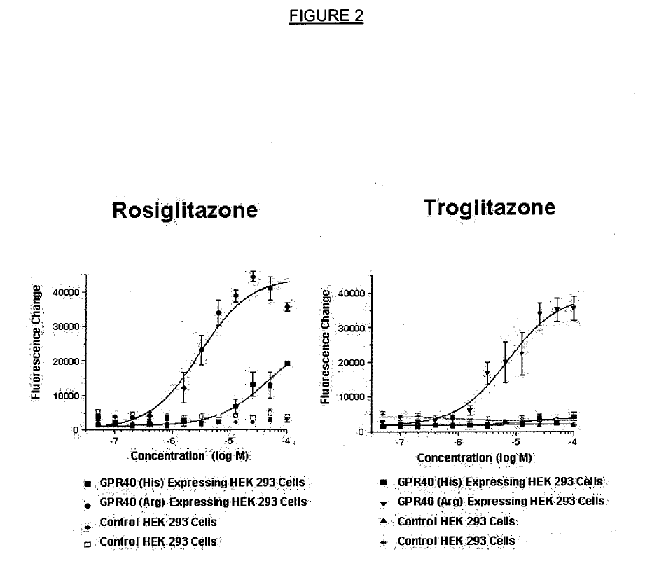

As illustrated by FIG. 2, cells in which nucleotide 632 of GPR40 is G have a higher response to, for example, thiazolidinedione activation, as compared to cells having the A nucleotide at position 632. FIG. 2 shows the results of a cell-based assay measuring intracellular calcium ion in cells having the G versus A nucleotide at position 632.

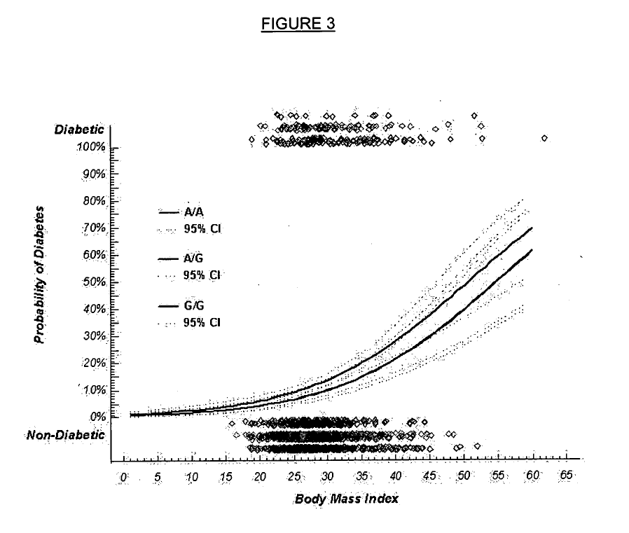

Furthermore, this invention is based, in part, on the discovery that individuals who have G at nucleotide 642, particularly those who have G on both alleles, have a higher risk for diabetes compared to individuals in which the nucleotide is A. As illustrated by FIG. 3, for a given body mass index subjects that are heterozygous or homozygous A at nucleotide 642 have a significantly lower chance of having Type II diabetes than a subject that is homozygous G.

The present invention encompasses characterization methods and methods of treatment or prevention of diseases or pathological conditions that are related to having at least one allele, preferably both alleles, of GPR40 wherein the encoded amino acid number 211 is other than histidine, preferably arginine. Genotyping of subjects for the methods of the invention may be performed by methods known to those of skill in the art based upon the present disclosure. Exemplary methods are described, for example, in Chapter 2 of Dracopoli et al. (2004).

Genotyping may be performed using an appropriate tissue of a subject as is known to those of skill in the art based upon the present disclosure. In a preferred embodiment, DNA for genotyping is isolated from whole a blood sample by procedures well known to those of skill in the art based upon the present disclosure. These procedures may be conducted using a variety of commercially available kits, such as, the Puregene® DNA Isolation Kit (Gentra Systems, Inc., Minneapolis, Minn.), DNA Isolation Kit for Blood (catalog no. 2-032-805, Roche Diagnostics Corporation), GenomicPrep™ Blood DNA Isolation Kit (catalog no. 27-5236-01, Amersham Biosciences Corp., Piscataway, N.J.), PAXgene Blood DNA Kit (catalog no. 761133, QIAGEN Inc., Valencia, Calif.), GNOME® Whole Blood DNA Isolation Kit (catalog no. 2011-600, Qbiogene, Inc., Carlsbad, Calif.) and Wizard® Genomic DNA Purification Kit (catalog no. A1120, Promega U.S., Madison, Wis.).

The nucleic acid region containing the single nucleotide polymorphism may be amplified, for example, by PCR techniques using a sense and an anti-sense primer and a detection probe. However, other nucleic acid amplification methods may also be used, including ligase chain reaction (see, for example, Abravaya, K. et al. (1995), branched DNA signal amplification (see, for example, Jrdea, M. S. et al. (1993)), isothermal nucleic acid sequence based amplification (NASBA) (see, for example, Kievits, T. et al. (1991)), and other self-sustained sequence replication assays.

Methods for preparing PCR primers and probes are well known in the art, and are described, for example, in Sambrook et al. (1989), Ausubel et al. (1994) and Innis et al. (1990). PCR primer pairs can be derived from known sequences, for example, by using computer programs intended for that purpose such as Primer (Version 0.5, 1991, Whitehead Institute for Biomedical Research, Cambridge Mass.).

Oligonucleotides for use as primers may be selected using software known in the art for such purposes. For example, OLIGO® version 6 software (Molecular Biology Insights, Inc., Cascade, Colo., www.oligo.net) is useful for the selection of PCR primer pairs of up to 100 nucleotides each. Similar primer selection programs have incorporated additional features for expanded capabilities. For example, the PrimOU primer selection program (available to the public from the Genome Center at University of Texas, South West Medical Center, Dallas Tex., ftp://ftp.qenome.ou.edu/pub/programs/primou_src.tar) is capable of choosing specific primers from megabase sequences and is thus useful for designing primers on a genome-wide scope. The Primer3, version 0.9, primer selection program (available to the public from the Whitehead Institute/MIT Center for Genome Research, Cambridge Mass., http://www-genome.wi.mit.edu/qenome_software/other/primer3.html) allows the user to input a “mispriming library,” in which sequences to avoid as primer binding sites are user-specified. Primer3 is useful, in particular, for the selection of oligonucleotides for microarrays. (The source code for the latter two primer selection programs may also be obtained from their respective sources and modified to meet the user's specific needs.) The PrimeGen program (available to the public from the UK Human Genome Mapping Project Resource Centre, Cambridge UK, http://www.hgmp.mrc.ac.uk/Registered/Option/primegen.html) designs primers based on multiple sequence alignments, thereby allowing selection of primers that hybridize to either the most conserved or least conserved regions of aligned nucleic acid sequences. Hence, this program is useful for identification of both unique and conserved oligonucleotides and polynucleotide fragments. Other oligonucleotide selection methods will be apparent to those of skill in the art based upon the present disclosure.

Preferably, the length of the sequence used for probes is minimized using a minor groove binder (MGB) linked to the probe sequence in order to discriminate between the two SNPs. MGBs are known to those of skill in the art, for example, as disclosed in U.S. Pat. Nos. 5,801,155; 6,084,102; 6,426,408; 6,312,894; and 6,683,173.

In a preferred embodiment, the Primer Express® version 1.5 software (Applied Biosystems, Foster City, Calif.) is used to design PCR primers and probes for the practice of the invention. Exemplary primers include CTTGGCCATCACAGCCTTCT for the forward primer (SEQ. ID. NO: 5) and CCACGTTGGAGGCGTTGT for the reverse primer (SEQ. ID. NO: 6). Probes may be FAM™ and VIC® dye-labeled TaqMan® MGB probes (Applied Biosystems) having the sequences 6FAM-CACTGGCCCACTCT and VIC-CACTGGCCCGCTC (SEQ. ID. Nos. 7 and 8 respectively). Isolated DNA of a subject is treated with the PCR reagents, including the primers and probes, and exposed to repeated thermal cycling in order to develop the PCR reaction. Detection of the SNP may be performed, for example, using a fluorescence based sequence detection system such as the ABI PRISM® 7900HT Sequence Detection System (AME Bioscience, Toroed, Norway).

The invention also encompasses diagnostic test kits for determining whether a subject has a nucleotide other than A, preferably G, at nucleotide 632 of the GPR40 coding sequence. For example, the kit can comprise a reagent such as a labeled or labelable nucleic acid probe capable of detecting whether a subject has A or G at nucleotide 632 of the GPR40 coding sequence. Such a probe is preferably less than 35 nucleotides in length, more preferably, less than 25 nucleotides and, even more preferably, between 13 and 25 nucleotides. The kit can further comprise reagents, such as PCR primers, for amplifying the nucleic acid region containing nucleotide 632. The reagents can be packaged in a suitable container. The kit can further comprise instructions for using the kit to detect whether a subject has A or G at nucleotide 632 of the GPR40 coding sequence.

The present invention provides methods for identifying test Agents that activate GPR40 by comparing activation by a test Agent of a first human GPR40 receptor wherein amino acid 211 is other than histidine, preferably arginine, to activation by a test Agent of a second human GPR40 receptor wherein amino acid 211 is histidine. Any appropriate method that measures activation of GPR40 may be used in the practice of this invention. Such methods are known or will be apparent to those of skill in the art based upon the present disclosure. For example, in a preferred embodiment, such methods involve the use of calcium sensitive dyes in a fluorometric imaging plate reader system (i.e., FLIPR®) which measure calcium flux following activation of G protein coupled receptors mediated through the Gq protein (see, for example, Chambers et al. (2003). Other methods known in the art include the use of calcium binding proteins (Miyawaki et al. (1997), Kain (1999)) and calcium sensitive luciferase (Button and Brownstein (1997), Stables et al. (1997)).

The invention further provides methods for identifying test Agents that inhibit activation of GPR40 by comparing inhibition of activation by a test Agent of a first human GPR40 receptor wherein amino acid 211 is histidine, to inhibition of a second human GPR40 receptor wherein amino acid 211 is other than histidine, preferably arginine. Such methods may utilize the activation assays described above using a known activator of GPR40 in combination with a test Agent. The detectable signal produced is compared to the signal that would be expected if the known activator were used by itself. Various activators of GPR40 are known in the art and may be used for such methods, including those described in Kotarsky et al. (2003), Briscoe et al. (2003) and International Patent Application Publication No. WO 02/057783.

This invention also encompasses methods for identifying test Agents that bind to GPR40, by comparing the competitive inhibition by a test Agent of binding of a known GPR40 ligand to a first human GPR40 receptor wherein amino acid 211 is histidine to competivive inhibition by the compound of binding the known ligand to a second human GPR40 receptor wherein amino acid 211 is other than histidine, preferably arginine. The ligand may be labeled with a detectable label as is well known in the art, including, for example, radioisotopes, fluorescent dyes and enzymes. Various ligands of GPR40 are known in the art and may be used for such methods, including those described in Kotarsky et al. (2003), Briscoe et al. (2003) and International Patent Application Publication No. WO 02/057783.

Those of skill in the art will appreciate, based upon the present disclosure, that the comparison of activation, inhibiton or activation and inhibition of binding of the methods for identifying test Agents of this invention may be performed by any of a number of ways. For example, the measurement of activation and inhibition of activation of GPR40 may be performed using fluorometric or colorimetric methods that measure calcium flux (e.g., FLIPR). Hence, activation of GPR40 may be accurately quantified by such methods, for example, by using a light detector instrument that generates a numerical value. The resulting values from such measurements may be compared by automated or manual means. For example, a practical method, particularly where large numbers of values have been generated, would involve comparing the values using a computerized system by methods well known to those with skill in the art.

Furthermore, the present invention encompasses methods of treatment or prevention of diseases or pathological conditions that are related to having at least one allele, preferably both alleles, of GPR40 wherein the encoded amino acid number 211 is other than histidine, preferably arginine. Methods of identifying such diseases or conditions are well known to those skilled in the art or will be apparent based upon the present disclosure. For example, FIG. 3 illustrates the results of a study comparing the genotype of subjects with Type II diabetes with subjects who do not have diabetes. It will be apparent to those skilled in the art that methods similar to those described in FIG. 3 and Example 5 may be employed to identify other diseases or conditions that are related to having at least one allele, preferably both alleles, of GPR40 wherein the encoded amino acid number 211 is other than histidine, preferably arginine.

In one embodiment of the present invention, a disease or pathological condition mediated by having at least one allele, preferably both alleles, of GPR40 wherein the encoded amino acid number 211 is other than histidine, preferably arginine is diabetes, preferably Type II diabetes. In another embodiment, the diseases or conditions are complications associated with diabetes. Such complications include arteriosclerosis, diabetic cardiomyopathy, cataracts, foot ulcers, diabetic macroangiopathy, diabetic microangiopathy, diabetic nephropathy, diabetic retinopathy and diabetic neuropathy. In a further embodiment, such diseases or conditions are any of the risk factors associated with metabolic syndrome, including obesity, hypertension, insulin resistance, Type II diabetes and dyslipidemia.

The present invention provides methods of treatment relating to the administration of an Agent for treatment or prevention of a disease or pathological condition mediated by having at least one allele, preferably both alleles, of the GPR40 gene wherein the encoded amino acid number 211 is other than histidine, preferably arginine (previously defined as the “Agents”). The Agents will be apparent to those skilled in the art based upon this disclosure or may be readily identified by methods known in the art. For example, the Agents may include insulin, insulin secretion stimulating sulfonylurea compounds, glycogen phosphorylase inhibitors (GPI), biguanide hepatic glucose output inhibitors, thiazolidinedione antidiabetic compound, alpha-glucosidase inhibitors, protein tyrosine phosphatase-1B (PTP-1B) inhibitors, dipeptidyl peptidase IV (DPPIV) inhibitors, glycogen synthase kinase-3 beta (GSK-3β) inhibitors, peroxisome proliferator-activated receptor gamma (PPARγ) agonists, glucagon receptor antagonists, selective serotonin reuptake inhibitors (SSRI's), 3-hydroxy-3-methylglutaryl coenzyme A reductase inhibitors (statins), γ-aminobutyric acid (GABA) agonists, angiotensin converting enzyme (ACE) inhibitors, angiotensin-II (A-II) receptor antagonists, phosphodiesterase type 5 (PDE-5) inhibitors, sorbitol dehydrogenase inhibitors (SDI) and aldose reductase inhibitors (ARI).

The Agents may include any protein tyrosine phosphatase-1B (PTP-1B) inhibitor. The term protein tyrosine phosphatase-1B inhibitor refers to any compound that inhibits the enzyme protein tyrosine phosphatase-1B. PTP-1B is believed to inhibit the ability of insulin to reduce blood sugar levels.

Exemplary PTP-1B inhibitors, assays for identifying such inhibitors and preferred dosage and methods of administration are disclosed in the following U.S. patents, International Patent Application publications and other publications: U.S. Pat. No. 6,251,936, U.S. Pat. No. 6,221,902, U.S. Pat. No. 6,057,316, U.S. Pat. No. 6,001,867, U.S. Pat. No. 5,753,687, WO 01/46203, WO 01/46204, WO 01/46206, WO 01/17516, WO 00/53583, WO 99/58518, WO 99/61435, WO 99/58521, WO 99/58522, WO 99/58514, WO 99/58520, WO 99/58519, WO 99/58511, WO 99/61410, WO 99/15529, Malamas et al. (2000A), Bleasdale et al. (2001), Wrobel et al. (1999), Malamas et al. (2000B), Wang et al. (1998), Andersen et al. (2000), Iversen et al., (2000), Wrobel et al. (2000) and Kees (1996).

The Agents may include any glucagon receptor antagonist. The term glucagon receptor antagonists refers to any compound that antagonizes the glucagon receptor, thus inhibiting the release of glucose induced by glucagon binding to the glucagon receptor. Such antagonism is readily determined by those skilled in the art according to assays known to those skilled in the art.

Exemplary glucagon receptor antagonists, assays for identifying such antagonists and preferred dosage and methods of administration are disclosed in the following U.S. patents, International Patent Application publications and other publications: U.S. Pat. No. 6,218,431, U.S. Pat. No. 5,138,090, WO 98/04528, WO 99/01423, WO 00/39088, WO 00/69810, WO 98/21957, WO 98/22109, WO 98/22108, WO 97/16442, Livingston et al. (1999), Madsen et al. (1998), de Laszlo et al. (1999), Chang et al. (2001), Cascieri et al. (1999), Ling et al. (2001) and Guillon et al. (1998).

The Agents may include any glycogen synthase kinase-3 beta (GSK-3β) antagonist. Exemplary GSK-3β antagonists, assays for identifying such antagonists and preferred dosage and methods of administration are disclosed in the following U.S. patents, International Patent Application publications and other publications: U.S. Pat. No. 6,057,286, WO 01/56567, WO 01/09106, WO 01/49709, WO 01/44246, WO 01/44206, WO 01/42224, WO 00/21927, WO 00/38675, WO 99/65897, WO 98/16528, Coghlan et al. (200), Smith et al. (2001), Cross et al. (2001) and Lochhead et al. (2001).

The Agents for the invention may include alpha-glucosidase inhibitors. Any alpha-glucosidase inhibitor may be used as an Agent in the invention. Exemplary alpha-glucosidase inhibitors include acarbose (also known as Precose®) and miglitol (also known as Glyset®), and analogs, derivatives, prodrugs and pharmaceutically acceptable salts of those alpha-glucosidase inhibitors.

The Agents may include insulin secretion stimulating sulfonylurea compounds. Any insulin secretion stimulating sulfonylurea compound may be used as an Agent for the invention. Exemplary insulin secretion stimulating sulfonylurea compounds include glipizide, also known as Glucotrol® and Glucotrol XL@, glimepiride (also known as Amaryl®), glyburide and chlorpropamide (also known as Diabinese®); and analogs, derivatives, prodrugs and pharmaceutically acceptable salts thereof. A preferred insulin secretion stimulating sulfonylurea compounds is glipizide.

The Agents may include biguanide hepatic glucose output inhibitors. Any biguanide hepatic glucose output inhibitor may be used as an Agent in the practice of the invention. An exemplary biguanide is metformin, also known as Glucophage®.

The Agents may include PPARγ agonists, including thiazolidinedione and non-thiazolidinedione compounds. PPARγ agonists increase insulin sensitivity in tissues important for insulin action such as adipose tissue, skeletal muscle, and liver.

Any PPARγ agonists may be used as an Agent in the practice of the invention. Exemplary PPARγ agonists include those described in the following U.S. patents: U.S. Pat. No. 4,340,605; U.S. Pat. No. 4,342,771; U.S. Pat. No. 4,367,234; U.S. Pat. No. 4,617,312; U.S. Pat. No. 4,687,777 and U.S. Pat. No. 4,703,052; and analogs, derivatives, prodrugs and pharmaceutically acceptable salts thereof. Preferred PPARγ agonists include darglitazone, ciglitazone, englitazone, pioglitazone, also known as Actos®, and rosiglitazone, also known as Avandia® and BRL-49653. PPARγ agonists are preferably administered in amounts ranging from about 0.1 mg/day to about 100 mg/day in single or divided doses, preferably about 0.1 mg/day to about 50 mg/day for an average subject, depending upon the thiazolidinedione antidiabetic compound and the route of administration. However, some variation in dosage will necessarily occur depending on the condition of the subject being treated. The individual responsible for dosing will, in any event, determine the appropriate dose for the individual subject.

The Agents may include any dipeptidyl peptidase IV (DPP IV) inhibitors. The term DPP IV inhibitor refers to any compound which inhibits the enzyme dipeptidyl peptidase. Such inhibition is readily determined by those skilled in the art according to assays such as those disclosed in International Patent Application publication number WO 98/19998.

Exemplary DPP IV inhibitors include those disclosed in U.S. patent Numbers U.S. Pat. No. 6,124,305, U.S. Pat. No. 6,110,949 and U.S. Pat. No. 6,124,305, in International Patent Application Publication Nos. WO 01/34594, WO 99/61431, WO 98/19998, WO 97/40832 and WO 95/15309 and in Augustyns et al. (1997); and analogs, derivatives, prodrugs and pharmaceutically acceptable salts thereof.

Preferred dosage and methods of administration are according to those provided in WO 01/34594 and WO 98/19998. Some variation in dosage may necessarily occur depending on the condition of the subject being treated. The individual responsible for dosing will, in any event, determine the appropriate dose for the individual subject.

The Agents may also comprise any selective serotonin reuptake inhibitor (SSRI). The term selective serotonin reuptake inhibitor refers to an compound which inhibits the reuptake of serotonin by afferent neurons. Such inhibition is readily determined by those skilled in the art according to standard assays such as those disclosed in U.S. Pat. No. 4,536,518 and other U.S. patents recited in the next paragraph.

Preferred SSRIs which may be used in accordance with this invention include femoxetine, which may be prepared as described in U.S. Pat. No. 3,912,743; fluoxetine, which may be prepared as described in U.S. Pat. No. 4,314,081; fluvoxamine, which may be prepared as described in U.S. Pat. No. 4,085,225; indalpine, which may be prepared as described in U.S. Pat. No. 4,064,255; indeloxazine, which may be prepared as described in U.S. Pat. No. 4,109,088; milnacipran, which may be prepared as described in U.S. Pat. No. 4,478,836; paroxetine, which may be prepared as described in U.S. Pat. No. 3,912,743 or U.S. Pat. No. 4,007,196; sertraline, which may be prepared as described in U.S. Pat. No. 4,536,518; sibutramine, which may be prepared as described in U.S. Pat. No. 4,929,629; and zimeldine, which may be prepared as described in U.S. Pat. No. 3,928,369. Fluoxetine is also known as Prozac®. Sertraline hydrochloride, also known as Zoloft®, may be prepared as set forth in U.S. Pat. No. 4,536,518. Sibutramine is also known as Meridia®. SSRIs that may be used as Agents include analogs, derivatives, prodrugs and pharmaceutically acceptable salts of the SSRIs described above.

SSRIs are preferably administered in amounts ranging from about 0.01 mg/kg/day to about 500 mg/kg/day in single or divided doses, preferably about 10 mg to about 300 mg per day for an average subject, depending upon the SSRI and the route of administration. However, some variation in dosage will necessarily occur depending on the condition of the subject being treated. The individual responsible for dosing will, in any event, determine the appropriate dose for the individual subject.

The Agents may further comprise any 3-hydroxy-3-methylglutaryl coenzyme A (HMG-CoA) reductase inhibitor (statin). The term 3-hydroxy-3-methylglutaryl coenzyme A (HMG-CoA) reductase inhibitor refers to a pharmaceutical compound which inhibits the enzyme 3-hydroxy-3-methylglutaryl-coenzyme A (HMG-CoA) reductase. This enzyme is involved in the conversion of HMG-CoA to mevalonate, which is one of the steps in cholesterol biosynthesis. Such inhibition is readily determined according to standard assays well known to those skilled in the art.

Preferred statins which may be used in accordance with this invention include atorvastatin, disclosed in U.S. Pat. No. 4,681,893, atorvastatin calcium, disclosed in U.S. Pat. No. 5,273,995, cerivastatin, disclosed in U.S. Pat. No. 5,502,199, dalvastatin, disclosed in European Patent Application Publication No. 738,510 A2, fluindostatin, disclosed in European Patent Application Publication No. 363,934 A1, fluvastatin, disclosed in U.S. Pat. No. 4,739,073, lovastatin, disclosed in U.S. Pat. No. 4,231,938, mevastatin, disclosed in U.S. Pat. No. 3,983,140, pravastatin, disclosed in U.S. Pat. No. 4,346,227, simvastatin, disclosed in U.S. Pat. No. 4,444,784 and velostatin, disclosed in U.S. Pat. No. 4,448,784 and U.S. Pat. No. 4,450,171. Especially preferred 3-hydroxy-3-methylglutaryl coenzyme A reductase inhibitors include atorvastatin, atorvastatin calcium, also known as Lipitor®, lovastatin, also known as Mevacor®, pravastatin, also known as Pravachol®, and simvastatin, also known as Zocor®; and analogs, derivatives, prodrugs and pharmaceutically acceptable salts thereof.

Statins are preferably administered in amounts ranging from about 0.1 mg/kg to about 1000 mg/kg/day in single or divided doses, preferably about 1 mg/kg/day to about 200 mg/kg/day for an average subject, depending upon the statin and the route of administration. However, some variation in dosage will necessarily occur depending on the condition of the subject being treated. The individual responsible for dosing will, in any event, determine the appropriate dose for the individual subject.

The Agents may include any angiotensin converting enzyme (ACE) inhibitor. The term angiotensin converting enzyme inhibitor refers to a pharmaceutical compound which inhibits angiotensin converting enzyme activity. ACE is involved in the conversion of angiotensin I to the vasoconstrictor, angiotensin II. The activity of ACE inhibitors may readily be determined by methods known to those skilled in the art, including any of the standard assays described in the patents listed below.

Preferred ACE inhibitors include: alacepril, disclosed in U.S. Pat. No. 4,248,883; benazepril, disclosed in U.S. Pat. No. 4,410,520; captopril, disclosed in U.S. Pat. Nos. 4,046,889 and 4,105,776; ceronapril, disclosed in U.S. Pat. No. 4,452,790; delapril, disclosed in U.S. Pat. No. 4,385,051; enalapril, disclosed in U.S. Pat. No. 4,374,829; fosinopril, disclosed in U.S. Pat. No. 4,337,201; imadapril, disclosed in U.S. Pat. No. 4,508,727; lisinopril, disclosed in U.S. Pat. No. 4,555,502; moexipril, disclosed in U.S. Pat. No. 4,344,949; moveltopril, disclosed in Belgian Patent No. 893,553; perindopril, disclosed in U.S. Pat. No. 4,508,729; quinapril, disclosed in U.S. Pat. No. 4,344,949; ramipril, disclosed in U.S. Pat. No. 4,587,258; spirapril, disclosed in U.S. Pat. No. 4,470,972; temocapril, disclosed in U.S. Pat. No. 4,699,905; and trandolapril, disclosed in U.S. Pat. No. 4,933,361; and analogs, derivatives, prodrugs and pharmaceutically acceptable salts thereof.

ACE inhibitors are preferably administered in amounts ranging from about 0.01 mg/kg/day to about 500 mg/kg/day in single or divided doses, preferably about 10 mg to about 300 mg per day for an average subject, depending upon the ACE inhibitor and the route of administration. However, some variation in dosage will necessarily occur depending on the condition of the subject being treated. The individual responsible for dosing will, in any event, determine the appropriate dose for the individual subject.

The Agents may include any angiotensin-II receptor (A-II) antagonist. The term angiotensin-II receptor antagonist refers to a pharmaceutical compound that blocks the vasoconstrictor effects of angiotensin II by blocking the binding of angiotensin II to the AT₁ receptor found in many tissues, (e.g., vascular smooth muscle, adrenal gland). The activity of an A-II antagonist may readily be determined by methods known to those skilled in the art, including any of the standard assays described in the patents listed below.

Preferred A-II antagonists include: candesartan, which may be prepared as disclosed in U.S. Pat. No. 5,196,444; eprosartan, which may be prepared as disclosed in U.S. Pat. No. 5,185,351; irbesartan, which may be prepared as disclosed in U.S. Pat. No. 5,270,317; losartan, which may be prepared as disclosed in U.S. Pat. No. 5,138,069; and valsartan, which may be prepared as disclosed in U.S. Pat. No. 5,399,578; and analogs, derivatives, prodrugs and pharmaceutically acceptable salts thereof. More preferred angiotensin-II receptor antagonists are losartan, irbesartan and valsartan.

A-II antagonists are preferably administered in amounts ranging from about 0.01 mg/kg/day to about 500 mg/kg/day in single or divided doses, preferably about 10 mg to about 300 mg per day for an average subject, depending upon the A-II antagonist and the route of administration. However, some variation in dosage will necessarily occur depending on the condition of the subject being treated. The individual responsible for dosing will, in any event, determine the appropriate dose for the individual subject.

The Agents may include any γ-aminobutyric acid (GABA) agonist. The term γ-aminobutyric acid agonist refers to a pharmaceutical compound that binds to GABA receptors in the mammalian central nervous system. GABA is the major inhibitory neurotransmitter in the mammalian central nervous system. The activity of a GABA agonist may readily be determined by methods known to those skilled in the art, including the procedures disclosed in Janssens de Verebeke, P. et al., Biochem. Pharmacol., 31, 2257-2261 (1982), Loscher, W., Biochem. Pharmacol., 31, 837-842, (1982) and/or Phillips, N. et al., Biochem. Pharmacol., 31, 2257-2261.

Preferred GABA agonists include: muscimol, which may be prepared as disclosed in U.S. Pat. No. 3,242,190; progabide, which may be prepared as disclosed in U.S. Pat. No. 4,094,992; riluzole, which may be prepared as disclosed in U.S. Pat. No. 4,370,338; baclofen, which may be prepared as disclosed in U.S. Pat. No. 3,471,548; gabapentin (Neurontin®), which may be prepared as disclosed in U.S. Pat. No. 4,024,175; vigabatrin, which may be prepared as disclosed in U.S. Pat. No. 3,960,927; tiagabine (Gabitril®), which may be prepared as disclosed in U.S. Pat. No. 5,010,090; lamotrigine (Lamictal®), which may be prepared as disclosed in U.S. Pat. No. 4,602,017; pregabalin, which may be prepared as disclosed in U.S. Pat. No. 6,028,214; phenyloin (Dilantin®), which may be prepared as disclosed in U.S. Pat. No. 2,409,754; carbamazepine (Tegretol®), which may be prepared as disclosed in U.S. Pat. No. 2,948,718; and topiramate (Topamax®) which may be prepared as disclosed in U.S. Pat. No. 4,513,006; and analogs, derivatives, prodrugs and pharmaceutically acceptable salts of those GABA agonists.

In general, in accordance with this invention, the GABA agonist used as the Agents will be administered in a dosage of about 4 mg/kg body weight of the subject to be treated per day to about 60 mg/kg body weight of the subject to be treated per day, in single or divided doses. However, some variation in dosage will necessarily occur depending upon the condition of the subject being treated. The person responsible for administration will, in any event, determine the appropriate dose for the individual subject. In particular, when used as the GABA agonist in this invention, pregabalin will be dosed at about 300 mg to about 1200 mg per day; gabapentin will be dosed at about 600 mg to about 3600 mg per day.

The Agents may include any glycogen phosphorylase inhibitor (GPI). The term glycogen phosphorylase inhibitor refers to any substance or compound or any combination of substances and/or compounds which reduces, retards, or eliminates the enzymatic action of glycogen phosphorylase. Such actions are readily determined by those skilled in the art according to standard assays as described in U.S. Pat. No. 5,988,463. U.S. Pat. No. 5,988,463, International Patent Application Publication No. WO 96/39384 and International Patent Application Publication No. WO 96/39385 exemplify GPI's that may be Agents.

GPIs are preferably administered in amounts ranging from about 0.005 mg/kg/day to about 50 mg/kg/day in single or divided doses, preferably about 0.1 mg/kg to about 15 mg/kg per day for an average subject, depending upon the GPI and the route of administration. However, some variation in dosage will necessarily occur depending on the condition of the subject being treated. The individual responsible for dosing will, in any event, determine the appropriate dose for the individual subject.

The Agents may include any sorbitol dehydrogenase inhibitor (SDI). The term sorbitol dehydrogenase inhibitor refers to any substance or compound or any combination of substances and/or compoundss which reduces, retards, or eliminates the enzymatic action of sorbitol dehydrogenase. Sorbitol dehydrogenase catalyzes the oxidation of sorbitol to fructose.

Exemplary SDIs include those disclosed in commonly assigned U.S. Pat. No. 5,728,704, U.S. Pat. No. 5,866,578 and International Patent Application Publication No. WO 00/59510; and analogs, derivatives, prodrugs and pharmaceutically acceptable salts thereof.

SDIs are preferably administered in amounts ranging from about 0.001 mg/kg/day to about 100 mg/kg/day in single or divided doses, preferably about 0.01 mg/kg to about 10 mg/kg per day for an average subject, depending upon the SDI and the route of administration. However, some variation in dosage will necessarily occur depending on the condition of the subject being treated. The person responsible for administration will, in any event, determine the appropriate dose for the individual subject.

The Agents may include any phosphodiesterase type 5 (PDE-5) inhibitor. The term phosphodiesterase type 5 inhibitor refers to any substance or compound or any combination of substances and/or compounds which reduces, retards, or eliminates the enzymatic action of cyclic guanosine monophosphate (c-GMP)-specific PDE-5. Such actions are readily determined by those skilled in the art according to assays as described in International Patent Application Publication No. WO 00/24745.

The following patent publications exemplify phosphodiesterase type 5 inhibitors which can be used as the Agents of this invention, and refer to methods of preparing those phosphodiesterase type 5 (PDE-5) inhibitors: International Patent Application Publication No. WO 00/24745; International Patent Application Publication No. WO 94/28902; European Patent Application Publication No. 0463756A1; European Patent Application Publication No. 0526004A1 and European Patent Application Publication No. 0201188A2. Preferred phosphodiesterase type 5 inhibitor are sildenafil (preferably sildenafil citrate, also known as Viagra®) which may be prepared as set forth in U.S. Pat. Nos. 5,250,534 and 5,955,611, tadalafil (also known as Cialis®) which may be prepared as set forth in U.S. Pat. No. 5,859,006 and vardinafil (also known as Levitra®) which may be prepared as set forth in U.S. Pat. No. 6,362,178. Exemplary PDE-5 inhibitors also include analogs, derivatives, prodrugs and pharmaceutically acceptable salts of the PDE-5 inhibitors listed above.

PDE-5 inhibitors are preferably administered in amounts ranging from about 5 mg/day to about 500 mg/day in single or divided doses, preferably about 10 mg/day to about 250 mg/day, for an average subject depending upon the PDE-5 inhibitor and the route of administration. However, some variation in dosage will necessarily occur depending on the condition of the subject being treated. The individual responsible for dosing will, in any event, determine the appropriate dose for the individual subject.

The Agents may include any aldose reductase inhibitor. The term aldose reductase inhibitor refers to compounds that inhibit the bioconversion of glucose to sorbitol catalyzed by the enzyme aldose reductase. Exemplary aldose reductase inhibitors include ponalrestat, disclosed in U.S. Pat. No. 4,251,528, tolrestat, disclosed in U.S. Pat. No. 4,600,724, epalrestat, disclosed in U.S. Pat. Nos. 4,464,382, 4,791,126 and 4,831,045, zenarestat, disclosed in U.S. Pat. Nos. 4,734,419, and 4,883,800, zopolrestat disclosed in U.S. Pat. No. 4,939,140 and preferred aldose reducatase inhibitors disclosed in U.S. Pat. No. 6,579,879. Exemplary aldose reductase inhibitors also include analogs, derivatives, prodrugs and pharmaceutically acceptable salts of the aldose reductase inhibitors listed above.

The amount of aldose reductase inhibitor that is administered per dose and the intervals between doses will depend upon the aldose reductase inhibitor being used, the type of pharmaceutical compositions being used, the characteristics of the subject being treated and the severity of the conditions, if any, being treated. Aldose reductase inhibitors are preferably administered in amounts ranging from about 0.001 mg/kg/day to about 1000 mg/kg/day in single or divided doses, preferably about 0.01 mg/kg to about 500 mg/kg per day for an average subject, depending upon the aldose reductase inhibitor and the route of administration. However, some variation in dosage will necessarily occur depending on the condition of the subject being treated. The person responsible for administration will, in any event, determine the appropriate dose for the individual subject. A preferred aldose reductase inhibitor is zopolrestat which is administered preferably at a dosage of between 250 mg and 500 mg per day.

The Agents may also include anti-obesity agents such as apolipoprotein-B secretion/microsomal triglyceride transfer protein (apo-B/MTP) inhibitors, 11β-hydroxy steroid dehydrogenase-1 (11β-HSD type 1) inhibitors, peptide YY₃₋₃₆ or analogs thereof, cannabinoid antagonists (e.g., CB-1 antagonists, such as rimonabant and purine compounds described in U.S. Patent Publication No. 2004/0092520), MCR-4 agonists, cholecystokinin-A (CCK-A) agonists, monoamine reuptake inhibitors (such as sibutramine), sympathomimetic agents, 3 adrenergic receptor agonists, dopamine agonists (such as bromocriptine), melanocyte-stimulating hormone receptor analogs, 5HT2c agonists, melanin concentrating hormone antagonists, leptin (the OB protein), leptin analogs, leptin receptor agonists, galanin antagonists, lipase inhibitors (such as tetrahydrolipstatin, i.e. orlistat), anorectic agents (such as a bombesin agonist), neuropeptide-Y receptor antagonists (e.g., NPY Y5 receptor antagonists, such as the spiro compounds described in U.S. Pat. Nos. 6,566,367, 6,649,624, 6,638,942, 6,605,720, 6,495,559, 6,462,053, 6,388,077, 6,335,345, and 6,326,375, U.S. Patent Publication Nos. 2002/0151456 and 2003/036652, and PCT Publication Nos. WO 03/010175, WO 03/082190 and WO 02/048152), thyromimetic agents, dehydroepiandrosterone or an analog thereof, glucocorticoid receptor agonists or antagonists, orexin receptor antagonists, glucagon-like peptide-1 receptor agonists, ciliary neurotrophic factors (such as Axokine™ available from Regeneron Pharmaceuticals, Inc., Tarrytown, N.Y. and Procter & Gamble Company, Cincinnati, Ohio), human agouti-related proteins (AGRP), ghrelin receptor antagonists, histamine 3 receptor antagonists or inverse agonists, neuromedin U receptor agonists and the like. Other anti-obesity agents, are well known, or will be readily apparent in light of the instant disclosure, to one of ordinary skill in the art. Especially preferred are anti-obesity agents selected from the group consisting of orlistat (prepared as described prepared as described in U.S. Pat. Nos. 5,274,143, 5,420,305, and 5,540,917), sibutramine (prepared as described prepared as described in U.S. Pat. No. 4,929,629), bromocriptine (U.S. Pat. Nos. 3,752,814 and 3,752,888), ephedrine, leptin, pseudoephedrine; rimonabant (prepared as described prepared as described in U.S. Pat. No. 5,624,941), peptide YY₃₋₃₆ or an analog thereof (prepared as described in U.S. Patent Publication No. 2002/0141985 and PCT Publication No. WO 03/027637); and and the NPY Y5 receptor antagonist 2-oxo-N-(5-phenylpyrazinyl)spiro-[isobenzofuran-1 (3H), 4′-piperidine]-1′-carboxamide. Other preferred NPY Y5 receptor antagonists are those described in PCT Publication No. WO 03/082190, including 3-oxo-N-(5-phenyl-2-pyrazinyl)-spiro[isobenzofuran-1 (3H), 4′-piperidine]-1′-carboxamide; 3-oxo-N-(7-trifluoromethylpyrido[3,2-b]pyridin-2-yl)-spiro-[isobenzofuran-1 (3H), 4′-piperidine]-1′-carboxamide; N-[5-(3-fluorophenyl)-2-pyrimidinyl]-3-oxospiro-[isobenzofuran-1 (3H), [4′-piperidine]-1′-carboxamide; trans-3′-oxo-N-(5-phenyl-2-pyrimidinyl)]spiro[cyclohexane-1,1′(3′H)-isobenzofuran]-4-carboxamide; trans-3′-oxo-N-[1-(3-quinolyl)-4-imidazolyl]spiro[cyclohexane-1,1′(3′H)-isobenzofuran]-4-carboxamide; trans-3-oxo-N-(5-phenyl-2-pyrazinyl)spiro[4-azaiso-benzofuran-1 (3H), 1′-cyclohexane]-4′-carboxamide; trans-N-[5-(3-fluorophenyl)-2-pyrimidinyl]-3-oxospiro[5-azaisobenzofuran-1 (3H), 1′-cyclohexane]-4′-carboxamide; trans-N-[5-(2-fluorophenyl)-2-pyrimidinyl]-3-oxospiro[5-azaisobenzofuran-1 (3H), 1′-cyclohexane]-4′-carboxamide; trans-N-[1-(3,5-difluorophenyl)-4-imidazolyl]-3-oxospiro[7-azaisobenzofuran-1 (3H), 1′-cyclohexane]-4′-carboxamide; trans-3-oxo-N-(1-phenyl-4-pyrazolyl)spiro[4-azaisobenzofuran-1 (3H), 1′-cyclohexane]-4′-carboxamide; trans-N-[1-(2-fluorophenyl)-3-pyrazolyl]-3-oxospiro[6-azaisobenzofuran-1 (3H), 1′-cyclohexane]-4′-carboxamide; trans-3-oxo-N-(1-phenyl-3-pyrazolyl)spiro[6-azaisobenzofuran-1 (3H), 1′-cyclohexane]-4′-carboxamide; trans-3-oxo-N-(2-phenyl-1,2,3-triazol-4-yl)spiro[6-azaisobenzofuran-1 (3H), 1′-cyclohexane]-4′-carboxamide, and and pharmaceutically acceptable salts and prodrugs thereof. Preferably, compounds of the present invention and combination therapies are administered in conjunction with exercise and a sensible diet.

The Agents are employed for the methods of this invention either alone or in combination with one or more other Agents. The Agents may be administered alone or with one or more pharmaceutically acceptable carriers, diluents or fillers. Pharmaceutical compositions containing the Agents may be readily administered in a variety of dosage forms such as tablets, powders, lozenges, syrups, injectable solutions and so forth. These pharmaceutical compositions may, if so desired, contain additional ingredients such as flavorings, binders, excipients and the like. For the purposes of oral administration, tablets containing various excipients such as sodium citrate, calcium carbonate, and calcium diphosphate may be used along with various disintegrants such as starch, alginic acid and certain complex silicates, together with binding agents such as polyvinylpyrrolidone, sucrose, gelatin and acacia. Additionally, lubricating agents such as magnesium stearate, sodium lauryl sulfate, and talc are often useful for tabletting purposes. Solid compositions of a similar type may also be used as fillers in soft and hard filled gelatin capsules. Preferred materials for this use include lactose or milk sugar and high molecular weight polyethylene glycols. When aqueous suspensions or elixirs are desired for oral administration, the Agent therein may be combined with various sweetening or flavoring agents, coloring matter or dyes and, if desired, emulsifying or suspending agents, together with diluents such as water, ethanol, propylene glycol, glycerin or various combinations thereof.

For parenteral administration, solutions of the Agents useful in this invention, in sesame or peanut oil, aqueous propylene glycol, or in sterile aqueous solution may be employed. Such aqueous solutions should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, the sterile aqueous media employed are all readily available by standard techniques known to those skilled in the art. Methods of preparing various pharmaceutical compositions with a certain amount of active ingredient are known, or will be apparent in light of this disclosure, to those with skill in the art. For examples, methods of preparing pharmaceutical compositions are described in Gennaro (2003).

The disclosures of all patents, applications, publications and documents, including brochures and technical bulletins, cited herein, are hereby expressly incorporated by reference in their entirety. It is believed that one skilled in the art can, based on the present description, including the examples, drawings, and appendant claims, utilize the present invention to its fullest extent.

It is believed that one skilled in the art can, using the present description, including the Examples, sequence listings and attendant claims, utilize the present invention to its fullest extent. The following Examples are to be construed as merely illustrative of the practice of the invention and not limitative of the remainder of the disclosure in any manner whatsoever.

### EXAMPLES

### Example 1

### GPR40 Expressing 293 Cells

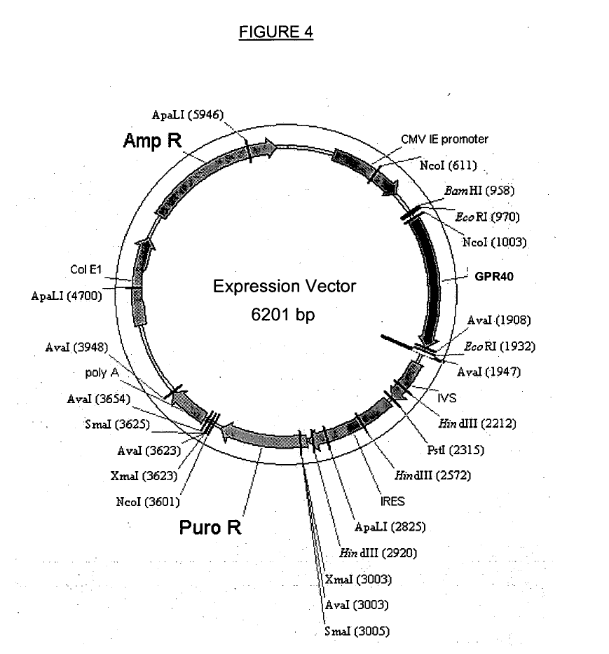

Expression vector constructs containing the GPR40 gene alleles were prepared based upon the vector, pIRESpuro, (catalog no. 6031-1, BD Biosciences, Franklin Lakes, N.J.). One such expression vector construct has the sequence of SEQ. ID. NO: 11 and the restriction map shown in FIG. 4. Human embryonic kidney (HEK) 293 cells which have been transformed with adenovirus 5 (available from the ATCC, catalog no. CRL-1573) were grown in media containing DMEM (catalog no. 11995-065, Invitrogen, Carlsbad, Calif.), 10% FBS (catalog no. 16140-071, Invitrogen) and 100 units Pen/Strep (catalog no. 15140-122, Invitrogen). When the cells reached 80% confluence, they were transfected with the GPR40 containing expression vectors using Fugene-6 reagent (Roche Diagnostics Corporation, Indianapolis, Ind.). Following 48 hours incubation, the media was aspirated and new media containing DMEM, 10% FBS, 100 units Pen/Strep and 1 g/ml puromycin were added. At four weeks, cloning cylinders (Sigma-Aldrich Co., St. Louis, Mo.) were used to select stable clones having puromycin resistance conveyed by the pIRESpuro vector. These colonies were allowed to amplify for an additional four weeks and fast growing colonies were selected resulting in HEK 293/GPR40 cells for use in the FLIPR assay of Example 2.

### Example 2

### GPR40 FLIPR Assay

On day one, HEK 293/GPR40 cells prepared according to Example 1 and control HEK 293 cells were plated in poly-D-lysine-coated 384-well black plates with clear bottom (catalog no. 354663, BD Biosciences) at 10,000 cells per well (30 ul per well of 3.3×10⁵ cells/mL solution) in growth medium containing DMEM, 10% FBS and Penn/Strep (100 μg/mL), with the addition of 1 μg/mL puromycin for HEK 293/GPR40 cells only, and incubated at 37° C. in 5% CO₂. On day two, the medium from each well was removed and replaced with 30 μl of serum-free medium (DMEM and Pen/Strep only, and no FBS), with the addition of 1 μg/mL puromycin for HEK 293/GPR40 cells only. On day three, dye vials of the FLIPR® Calcium 3 assay kit (catalog no. R8091, Molecular Devices, Sunnyvale Calif.) were reconsitituted with 11 mL Hanks' Buffered Saline Solution (component of FLIPR® assay kit) and 225 μl freshly-made 250 mM probenecid stock containing 5N NaOH (1 mL), probenecid (0.74 g) (catalog no. P8761, Sigma-Aldrich Co.) and 1×FLIPR buffer (9 mL) (made from 10×FLIPR buffer containing, per liter, NaCl (84.68 g), D(+) glucose anhydrous (18.02 g), MgSO₄.7H₂O (1.2 g), KCl (3.73 g), HEPES (23.8 g) and CaCl₂ (1.11 g), at pH 7.4). Fatty acids and other GPR40 ligand test compounds were diluted to concentrations of between 0.05 μM and 100 μM in FLIPR/DMSO (1×FLIPR buffer containing 0.25% DMSO). Thirty microliters of dye was added to each plate well and the plates were incubated at 37° C. and 5% CO₂ for one hour. Calcium flux was measured using a FLIPR plate reader (Molecular Devices). Test compound solutions were injected into each well (15 μl per well) at a rate of 10 μl per second. Exposures were made very two seconds for 0.4 seconds per exposure for 90 exposures.

### Example 3

### DNA Isolation and Purification

DNA from a whole blood sample is isolated and purified using the Puregene® DNA Isolation Kit (Gentra Systems, Inc., Minneapolis, Minn.) according to the manufacturer's instructions. Alternatively, red bloods cells from frozen whole blood samples may be lysed by mixing thawed whole blood (1 mL) with RBC Lysis Solution (Gentra Systems, Inc.), for example, in a 3:1 ratio (i.e., 3 mL RBC Lysis Solution to 1 mL whole blood) and mixed. The solution is incubated for 10 minutes at room temperature. After incubation, the solution is centrifuged at 1,800×g for 10 minutes. The supernatant from the tube is poured off and the resulting pellet is re-suspended in the residual supernatant. Cell Lysis Solution (1 mL) (Gentra Systems, Inc.) is then added, for example, to a volume equivalent to that of the initial whole blood volume. RNase A Solution (5 μl) (Gentra Systems, Inc.) is added to the cell lysate and the tube is incubated at 37° C. for 15 minutes. The sample is then cooled to room temperature, Protein Precipitation Solution (333 μl) (Gentra Systems, Inc.) is added to the cell lysate and the mixture is mixed by vortexing at high speed for at least 20 seconds. The mixture is then centrifuged at 1,800×g for 10 minutes resulting in a tight protein pellet. The resulting supernatant containing DNA is poured into a clean 15 mL centrifuge tube containing a 10 mL of 100% isopropanol (2-propanol). The sample is mixed by gently inverting the tube (approximately 40-60 times) until DNA precipitates. If DNA does not appear, seven μl of Glycogen (20 μg/mL) per mL of whole blood (original starting material) is added and incubate at room temperature for 15 minutes. The sample is then centrifuged at 1,800×g for three minutes resulting in a small white DNA pellet. The supernatant is poured off and the tube drained onto absorbent paper. Ten mL of 70% ethanol are added and the tube inverted several times to wash the DNA pellet. The sample is centrifuged for one minute at 1,800×g. The supernatant is then be poured off, and the tube is inverted and air-dried.

### Example 4

### Allelic Discrimination

Primers and Taqman® MGB probes (Applied Biosystems) were designed using the software Primer Express version 1.5 (Applied Biosystems). The PCR reaction was carried out in a volume of 5 μl consisting of: 2.5 μl of 2× TaqMan® Universal PCR Master Mix (no AmpErase), 200 nM of each probe, 900 nM of the forward and reverse primers, and water to 5 μl. Alternatively, primers and probes were obtained as a TaqMan Assays-on Demand™ (Applied Biosystems) and the PCR reaction was carried out in 5 μl consisting of: 2.5 μl of 2× TaqMan® Universal PCR Master Mix (no AmpErase), 2.25 μl water and 0.25 μl of 20× Assay Mix. The 5 μl reaction mixture was added to approximately 10 ng of dried DNA in the well of a 96 well plate. The plates were placed on a titer plate shaker and shaken vigorously for 10 minutes and then briefly spun at 1000 RPM. Thermal cycling was performed in a GeneAmp® PCR System 9700 (Applied Biosytems) with a dual 384-well sample block module using the following thermal cycling conditions: 950° C. for 10 minutes, 40 cycles of 92° C. for 15 seconds and 60° C. for one minute. The fluorescent signal was detected using an ABI PRISM 7900HT Sequence Detector (Applied Biosystems) according to the manufacture's instructions. The data was analyzed with the Sequence Detection System (SDS) software version 2.1 (Applied Biosytems).

### Example 5

### Investigation of Variants for GPR40 in Type II Diabetic Patients

Of the 2465 subjects analysed, 1332 were male and 857 were female. Allele frequencies were estimated by counting the occurrences of the particular allele of GRP40, and then dividing by two times number of individuals to account for the two alleles contributed by each subject. Genotype frequencies were estimated by counting the occurrences of a particular genotype and then dividing by the number of individuals.

### REFERENCES

Abravaya, K. et al. (1995), Nucleic Acids Research, 23, 675-682;
ADA (2004), “Standards of medical care in diabetes (Clinical Practice Recommendations 2004)”, Diabetes Care, 27 (Suppl 1), S15-S35;
Andersen, H S et al. (2000), “2-(oxalylamino)-benzoic acid is a general, competitive inhibitor of protein-tyrosine phosphatases”, J. Biol. Chem., 275, 7101-7108;
Armitage, P. (1955), “Tests for linear trends in proportions and frequencies”, Biometrics, 11, 375-386;
Augustyns, K J L et al., Eur. J. Med. Chem., 32, 301-309;
Ausubel et al. (1994), Current Protocols in Molecular Biology, John Wiley & Sons, Hoboken, N.J.;
Bleasdale, J E, et al. (2001), “Small molecule peptidomimetics containing a novel phosphotyrosine bioisostere inhibit protein tyrosine phosphatase 1B and augment insulin action”, Biochemistry, 40, 5642-5654;
Briscoe, C. P. et al. (2003), “The orphan G protein-coupled receptor GPR40 is activated by medium and long chain fatty acids”, J. Biol. Chem., 278, 11303-11311,
Button, D. and Brownstein, M. (1997), “Aequorin-expressing mammalian cell lines used to report Ca2+ mobilization”, Cell Calcium, 14, 663-671;
Cascieri, M A et al. (1999), “Characterization of a novel, non-peptidyl antagonist of the human glucagon receptor”, J. Biol. Chem., 274, 8694-8697;
Chambers, C. et al. (2003), “Measuring Intracellular Calcium Fluxes in High Throughput Mode”, Combinatorial Screening and High Throughput Screening, 6, 355-362;
Chang, L L et al. (2001), “Substituted imidazoles as glucagon receptor antagonists”, Bioorg. & Med. Chem. Lett., 11, 2549-2553;
Coghlan, M P et al. (2000), Chemistry and Biology, 7, 793-803;
Cross, D A et al. (2001), “Selective small-molecule inhibitors of glycogen synthase kinase-3 activity protect primary neurones from death”, Journal of Neurochemistry, 77, 94-102;
de Laszlo, S E et al. (1999), “Potent, orally absorbed glucagon receptor antagonists”, Bioorg. & Med. Chem. Lett., 9, 641-646;
Devlin, B. and Roeder, K. (1999), “Genomic control for association studies”, Biometrics, 55, 997-1004);
Dracopoli et al. (2004), Current Protocols in Human Genetics, John Wiley & Sons, Hoboken, N.J.;
Expert Panel on Detection, Evaluation, and Treatment of High Blood Cholesterol in Adults (2001), “Executive Summary of the Third Report of the national Cholesterol Eductation Program (NCEP),” JAMA 285, 2486-2497;
Gadsby, R. (2002), “Epidemiology of diabetes”, Adv. Drug Deliv. Rev. 54, 1165-1172;
Gether, U. (2000), “Uncovering molecular mechanisms involved in activation of G protein coupled receptors”, Endocr. Rev., 21, 90-113;
Guillon, J. et al. (1998), European Journal of Medicinal Chemistry, 33, 293-308;
Haber, E. P., et al. (2002), “Pleiotropic effects of fatty acids on pancreatic β-cells”, J. Cell Physiol., 194, 1-12;
Haga, H., et al. (2002), “Gene-based SNP discovery as part of the Japanese Millennium Genome Project: identification of 190,562 genetic variations in the human genome”, J. Hum. Genet., 47, 605-610;
Higuchi, T. and Stella, W. (1987), “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press;
Innis et al. (1990) PCR Protocols, A Guide to Methods and Applications, Academic Press, San Diego Calif.;
Itoh, Y., et al. (2003), “Free fatty acids regulate insulin secretion from pancreatic β cells through GPR40”, Nature, 173-176;
Iversen, L F et al., (2000) “Structure-based design of a low molecular weight, nonphosphorus, nonpeptide, and highly selective inhibitor of protein-tyrosine phosphatase 1B”, J. Biol. Chem., 275, 10300-10307;
Johnson, G. L. and Dhanasekaran, N. (1989), “The G-protein family and their interaction with receptors”, Endocr. Rev., 10, 317-331;
Jrdea, M S et al. (1993), AIDS, 7 (supp. 2), S11-514;
Kain, S. R. (1999), “Green fluorescent protein (GFP): applications in cell-based assays for drug discovery”, Drug Discov. Today, 4, 304-312;
Kievits, T. et al. (1991), J. Virological., Methods 35, 273-286;
Kees, K L, et al. (1996), “New potent antihyperglycemic agents in db/db mice: synthesis and structure-activity relationship studies of (4-substituted benzyl) (trifluoromethyl)pyrazoles and -pyrazolones”, J. Med. Chem., 39, 3920-3928;
Kotarsky, K. et al. (2003), “A human cell surface receptor activated by free fatty acids and thiazolidinedione drugs”, Biochem. Biophys. Res. Commun., 301, 406-410;
Ling, A et al. (2001), “Identification of alkylidene hydrazides as glucagon receptor antagonists”, J. Med. Chem., 44, 3141-3149;
Livingston, et al. (1999), Diabetes, 48 Suppl. 1, 0862;
Lochhead, P A et al. (2001), “Inhibition of GSK-3 selectively reduces glucose-6-phosphatase and phosphatase and phosphoenolypyruvate carboxykinase gene expression”, Diabetes, 50, 937-946;
Madsen, P. et al. (1998), “Discovery and structure-activity relationship of the first non-peptide competitive human glucagon receptor antagonists”, J. Med. Chem., 41, 5150-5157;
Malamas, M S, et al. (2000A), “New azolidinediones as inhibitors of protein tyrosine phosphatase 1B with antihyperglycemic properties”, J. Med. Chem., 43, 995-1010;
Malamas, M S et al. (2000B), “Novel benzofuran and benzothiophene biphenyls as inhibitors of protein tyrosine phosphatase 1B with antihyperglycemic properties”, J. Med. Chem., 43, 1293-1310;
Miyawaki, A. et al. (1997), “Fluorescent indicators for Ca2+ based on green fluorescent proteins and calmodulin”, Nature, 388, 882-887;
Park, Y W et al. (2003), “The Metabolic Syndrome. Prevalence and associated risk factor findings in the U.S. population from the Third National and Nutrition Examination Survey, 1988-1994”, Arch. Intern. Med., 163, 427-436;
Gennaro, A R (2003) “Remington: The Science and Practice of Pharmacy”, Lippincott Williams & Wilkins, Philadelphia, Pa., 20th Edition;
Reilly, MP and Rader, DJ (2003), “The Metabolic syndrome. More than the sum of its parts?”, Circulation, 108, 1546-1551;
Sambrook et al. (1989) Molecular Cloning: A Laboratory Manual, Cold Spring Harbor Press, Plainview N.Y.;
Sawzdargo, M. et al. (1997), “A cluster of four novel human G protein-coupled receptor genes occurring in close proximity to CD22 gene on chromosome 19q13.1”, Biochem. Biophys. Res. Commun., 239, 543-547;
Shirai, K. (2004), “Obesity as the core of the metabolic syndrome and the management of coronary heart disease”, Curr. Med. Res. Opin., 20, 295-304;
Smith, D G et al. (2001), “3-Anilino-4-arylmaleimides: potent and selective inhibitors of glycogen synthase kinase-3 (GSK-3)”, Bioorg. & Med. Chem. Lett., 11, 635-639;
Stables, J. et al. (1997), “A bioluminescent assay for agonist activity at potentially any G-protein-coupled receptor”, Anal. Biochem., 252, 115-126;
Wang, Q. et al. (1998), “Naphthalenebis[alpha,alpha-difluoromethylene phosphonates] as potent inhibitors of protein tyrosine phosphatases”, Bioorg. Med. Chem. Left., 8, 345-350;
Weir, BS and Cockerham, CC (1979), “Estimation of linkage disequilibrium in randomly mating populations”, Heredity, 42, 105-111;
Wrobel, J., et al. (1999), “PTP1B inhibition and antihyperglycemic activity in the ob/ob mouse model of novel 11-arylbenzo[b]naphtho[2,3-d]furans and 11-arylbenzo[b]naphtho[2,3-d]thiophenes”, J. Med. Chem., 42: 3199-3202;
Wrobel, J. et al. (2000), “Synthesis and PTP1B inhibition of novel 4-aryl-1-oxa-9-thiacyclopenta[b]fluorenes”, Bioorg. & Med. Chem. Left., 10, 1535-1538.

1. A genotyping method, comprising determining the amino acid that is encoded by the GPR40 gene of a human subject at amino acid number 211.

2. A method of claim 1 further comprising determining the amino acid that is encoded at amino acid number 211 by both alleles of the GPR40 gene of said subject.

3. A genotyping method of claim 1 further comprising determining whether the GPR40 gene of a said subject encodes an amino acid number 211 that is other than histidine.

4. A genotyping method of claim 1 further comprising determining whether the GPR40 gene of a said subject encodes an amino acid number 211 that is arginine.

5. A genotyping method of claim 1 further comprising determining whether the GPR40 gene of a said subject encodes an amino acid number 211 that is histidine.

6. A genotyping method of claim 1 further comprising:
isolating nucleic acid from a human subject;
amplifying a contiguous sequence of said nucleic acid, wherein said contiguous sequence comprises the nucleotides of GPR40 that encode amino acid number 211, or the complementary sequence thereof;
treating said contiguous sequence with a nucleotide probe that enables determination of the amino acid encoded at amino acid number 211 of the GPR40 gene of said subject; and
determining whether said probe hybridizes to said contiguous sequence under conditions of high stringency.

7. A method of claim 6 wherein said probe enables determination whether amino acid number 211 of said GPR40 gene is histidine.

8. A method of claim 6 wherein said probe enables determination whether amino acid number 211 of said GPR40 gene is other than histidine.

9. A method of claim 6 wherein said probe enables determination whether amino acid 211 of said GPR40 gene is arginine.

10. A method of claim 6 wherein said probe consists of a contiguous portion of a nucleotide sequence of SEQ. ID. NO: 9 having a length of about 13 to about 35 nucleotides, linked to a detectable label, and including the nucleotides encoding amino acid 211, or the complementary nucleotide sequence thereof.

11. A method of claim 6 wherein said probe consists of a contiguous portion of a nucleotide sequence of SEQ. ID. NO: 10 having a length of about 13 to about 35 nucleotides, linked to a detectable label, and including the nucleotides encoding amino acid 211, or the complementary nucleotide sequence thereof.

12. A method of treatment, comprising:
determining the amino acid that is encoded by the GPR40 gene of a human subject at amino acid number 211; and
treating said subject with a therapeutically effective amount of an Agent for treatment or prevention of a disease or pathological condition mediated by having at least one allele of the GPR40 gene wherein the encoded amino acid number 211 is other than histidine.

13. A method of claim 12 wherein said Agent is selected from:
insulin, an insulin secretion stimulating sulfonylurea compound, a glycogen phosphorylase inhibitor, a biguanide hepatic glucose output inhibitor, an alpha-glucosidase inhibitor, a protein tyrosine phosphatase-1B inhibitor, a dipeptidyl peptidase IV inhibitor, a glycogen synthase kinase-3 beta inhibitor, a peroxisome proliferator-activated receptor gamma agonist, a glucagon receptor antagonist, a selective serotonin reuptake inhibitor, a 3-hydroxy-3-methylglutaryl coenzyme A reductase inhibitor, a gamma-aminobutyric acid agonist, an angiotensin converting enzyme inhibitor, an angiotensin-II receptor antagonist, a phosphodiesterase type 5 inhibitor, a sorbitol dehydrogenase inhibitor and an aldose reductase inhibitor, a pharmaceutically acceptable prodrug thereof or pharmaceutically acceptable salt thereof.

14. A method of claim 12, wherein said Agent is a thiazolidinedione antidiabetic compound, a pharmaceutically acceptable prodrug thereof or a pharmaceutically acceptable salt thereof.

15. A method of claim 12, wherein said Agent is a dipeptidyl peptidase IV inhibitor, a pharmaceutically acceptable prodrug thereof or a pharmaceutically acceptable salt thereof.

16. A method of claim 12, wherein said Agent is a glycogen phosphorylase inhibitor, a pharmaceutically acceptable prodrug thereof or a pharmaceutically acceptable salt thereof.

17. A method of claim 12, wherein said Agent is an aldose reductase inhibitor, a pharmaceutically acceptable prodrug thereof or a pharmaceutically acceptable salt thereof.

18. A method of claim 12, wherein said Agent is a peroxisome proliferator-activated receptor gamma agonist, a pharmaceutically acceptable prodrug thereof or a pharmaceutically acceptable salt thereof.

19. A method of claim 12 wherein said Agent is an anti-obesity agent.

20. A method of claim 12 wherein said Agent is selected from:
an apolipoprotein-B secretion/microsomal triglyceride transfer protein inhibitor, an 11β-hydroxy steroid dehydrogenase-1 inhibitor, a peptide YY₃₋₃₆, an analog of a peptide YY₃₋₃₆, a cannabinoid antagonist, an MCR-4 agonist, a cholecystokinin-A agonist, a monoamine reuptake inhibitor, a sympathomimetic agents, a 3 adrenergic receptor agonist, a dopamine agonist, a melanocyte-stimulating hormone receptor analog, a 5HT2c agonist, a melanin concentrating hormone antagonist, leptin, a leptin analog, a leptin receptor agonist, a galanin antagonist, a lipase inhibitor, an anorectic agent, a neuropeptide-Y receptor antagonist, a thyromimetic agent, dehydroepiandrosterone, an analog of dehydroepiandrosterone, a glucocorticoid receptor agonist, a glucocorticoid receptor antagonist, an orexin receptor antagonist, a glucagon-like peptide-1 receptor agonist, a ciliary neurotrophic factor, a human agouti-related protein, a ghrelin receptor antagonist, a histamine 3 receptor antagonist, a histamine 3 receptor inverse agonist and a neuromedin U receptor agonist.

21. A kit, comprising a nucleotide probe that enables determination of the amino acid encoded at amino acid number 211 of the GPR40 gene of a human subject.

22. A kit of claim 21 wherein said probe enables determination whether amino acid number 211 of said GPR40 gene is histidine.

23. A kit of claim 21 wherein said probe enables determination whether amino acid number 211 of said GPR40 gene is other than histidine.

24. A kit of claim 21 wherein said probe enables determination whether amino acid 211 of said GPR40 gene is arginine.

25. A kit of claim 21 wherein said probe consists of a contiguous portion of a nucleotide sequence of SEQ. ID. NO: 9 having a length of about 13 to about 35 nucleotides, linked to a detectable label, and including the nucleotides encoding amino acid 211, or the complementary nucleotide sequence thereof.

26. A kit of claim 21 wherein said probe consists of a contiguous portion of a nucleotide sequence of SEQ. ID. NO: 10 having a length of about 13 to about 35 nucleotides, linked to a detectable label, and including the nucleotides encoding amino acid 211, or the complementary nucleotide sequence thereof.

27. A kit of claim 21, further comprising either: a first polymerase chain reaction nucleotide primer that is complementary to a nucleotide sequence that is downstream from the nucleotides encoding amino acid 211 of said GPR40 gene and a second polymerase chain reaction primer that is complementary to a nucleotide sequence that is upstream from the nucleotides encoding said amino acid 211; or, a first primer that is complementary to a nucleotide sequence that is upstream from the nucleotides encoding said amino acid 211 and a second polymerase chain reaction primer that is complementary to a nucleotide sequence that is downstream from the nucleotides encoding said amino acid 211.

28. A method of characterizing an Agent, comprising:
measuring the competitive inhibition by a test Agent of binding of a GPR40 ligand to a first human GPR40 receptor wherein amino acid number 211 is histidine; and
measuring the competitive inhibition by said compound of binding of a GPR40 ligand to a second human GPR40 receptor wherein amino acid number 211 is other than histidine.

29. A method of claim 28, wherein the amino acid number 211 of said second receptor is arginine.

30. A method of claim 28 or 29, further comprising comparing the competitive inhibition of binding by said compound of said first receptor and said second receptor.

31. A method of characterizing an Agent, comprising:
measuring the activation by a test Agent of a first human GPR40 receptor wherein amino acid number 211 is histidine; and
measuring the activation by a test Agent of a second human GPR40 receptor wherein amino acid number 211 is other than histidine.

32. A method of claim 31, wherein the amino acid number 211 of said second receptor is arginine.

33. A method of claim 31 or 32, further comprising comparing the activation by said compound of said first receptor and said second receptor.

34. A method of characterizing an Agent, comprising:
measuring the inhibition by a test Agent of activation of a first human GPR40 receptor by an activator of GPR40, wherein amino acid number 211 of said first receptor is histidine; and
measuring the inhibition by a test Agent of activation of a second human GPR40 receptor by an activator of GPR40, wherein amino acid number 211 of said second receptor is other than histidine.

35. A method of claim 34, wherein the amino acid number 211 of said second receptor is arginine.

36. A method of claim 34 or 35, further comprising comparing the inhibition of activation by said compound of said first receptor and said second receptor.


SEQUENCE LISTING (6 sequences)

SEQ ID NO: 1 - DNA, 27 bp, Rattus rattus
aaactccatcatatactcctgcagact

SEQ ID NO: 2 - DNA, 36 bp, Rattus rattus
attcttacagtgtgattattccaggcctcctctgag

SEQ ID NO: 3 - DNA, 20 bp, Rattus rattus
ttctctgcggcttcctcaaa

SEQ ID NO: 4 - DNA, 19 bp, Rattus rattus
gtgctgactgtggctttcc

SEQ ID NO: 5 - DNA, 32 bp, Rattus rattus
tcaaagattgaatcctgtagttcttcattgat

SEQ ID NO: 6 - DNA, 19 bp, Rattus rattus
ccagtttggacatagattc